(19) United States
(12) Patent Application Publication
(10) Pub. No.: US 2002/0131941 A1
(43) Pub. Date: Sep. 19, 2002

(54) Colorant-containing aqueous polymer dispersion

(76) Inventors: Thorsten Habeck, Meckenheim (DE); Klemens Mathauer, Ludwigshafen (DE); Thomas Wunsch, Speyer (DE); Horst Westenfelder, Neustadt (DE); Hideyuki Ichihara, Kanagawa (JP); Takahiro Ikeda, Yokkaichi-shi (JP); Wilma M. Dausch, Limburgerhof (DE)

Correspondence Address: KEIL & WEINKAUF, 1350 CONNECTICUT AVENUE, N.W., WASHINGTON, DC, 20036, US

(21) Appl. No.: 09/954,261

(22) Filed: Sep. 18, 2001

(30) Foreign Application Priority Data
Sep. 21, 2000 (DE) 10046927.2

(51) Int. Cl. A61K 7/021; A61K 7/42

(52) U.S. Cl. 424/63; 424/59

(57) ABSTRACT

The present invention relates to colorant-containing polymer dispersions comprising

a. colorant-containing polymer particles PC having an average particle diameter d₅₀ below 1000 nm, comprising:

i. a polymer matrix constructed from ethylenically unsaturated monomers M and

ii. at least one organic colorant C homogeneously distributed within the polymer matrix and chosen from dyes, UV absorbers and optical brighteners, in an amount of from 0.5 to 50% by weight, based on the polymer matrix;

b. at least one nonionic surface-active compound NS in an amount of from 0.1 to 20% by weight, based on the polymer matrix; and

c. at least one amphiphilic polymer PA which has 0.5 to 10 mol/kg of anionic functional groups, in an amount of from 1 to 50% by weight, based on the polymer matrix.

The invention also relates to a process for their preparation and to cosmetic compositions which comprise this dispersion or polymer powder prepared therefrom.

The present invention relates to colorant-containing aqueous polymer dispersions of finely divided, colorant-containing polymer particles and colorant-containing polymer powders prepared therefrom, and to the use thereof in cosmetic compositions.

Colorant-containing polymer dispersions and polymer powders prepared therefrom are known from WO 99/01967. They comprise at least one colorant which is present in the polymer particles of the colorant-containing polymer dispersion in largely homogeneous distribution.

In the colorant-containing polymers of WO 99/01967, the colorant is encapsulated by the polymer matrix. These colorant-containing polymers therefore exhibit similar advantages to an insoluble pigment, i.e. the encapsulated colorant is largely inert toward external influences, e.g. toward photooxidative decomposition or toward bleeding in the case of action of a solvent. Compared with pigments, the colorant-containing polymers are characterized by higher color brilliance. Moreover, the colorant-containing polymers are frequently characterized by easier handling and better compatibility with the application-related preparations compared with pigments.

The preparation of the colorant-containing polymers of WO 99/01967 is carried out by polymerization of an aqueous colorant-containing monomer emulsion. To stabilize the monomer droplets in the emulsion, use is usually made of at least one anionic emulsifier. The anionic emulsifiers customarily used for this purpose, however, have the disadvantage that they may lead to skin irritations. This is unacceptable particularly with regard to the use of these colorants. Replacement of these emulsifiers is therefore desirable.

However, the applicant's own investigations have shown that if these anionic emulsifiers are replaced by nonionic emulsifiers, the preparation of the colorant-containing polymers is not possible due to instabilities of the monomer emulsions to be polymerized. Replacement of the anionic emulsifiers by neutral or anionic protective colloids likewise did not lead to success.

Surprisingly, we have now found that colorant-containing aqueous polymer dispersions of finely divided, colorant-containing polymer particles can be prepared even in the absence of anionic emulsifiers if the polymerization is carried out in the presence of a surface-active stabilizer system which comprises a suitable amount of at least one nonionic surface-active compound NS and a suitable amount of at least one amphiphilic polymer PA which has 0.5 to 10 mol/kg of anionic functional groups.

Accordingly, the present invention firstly relates to colorant-containing aqueous polymer dispersions comprising:

a. colorant-containing polymer particles PC having a weight-average particle diameter d₅₀ below 1000 nm, comprising:

i. a polymer matrix constructed from ethylenically unsaturated monomers M and

ii. at least one organic colorant C homogeneously distributed within the polymer matrix and chosen from dyes, UV absorbers and optical brighteners, in an amount of from 0.5 to 50% by weight, based on the polymer matrix;

b. at least one nonionic surface-active compound NS in an amount of from 0.1 to 20% by weight, based on the polymer matrix; and

c. at least one amphiphilic polymer PA which has 0.5 to 10 mol/kg of anionic functional groups, in an amount of from 1 to 50% by weight, based on the polymer matrix.

The invention also relates to a process for the preparation thereof, the polymer powders obtainable from the colorant-containing aqueous polymer dispersions and cosmetic compositions which comprise the colorant-containing aqueous polymer dispersions according to the invention and/or the powders prepared therefrom. An important advantage of the colorants according to the invention is that they can be prepared in the absence of anionic emulsifiers and thus do not have to contain anionic emulsifiers, i.e. the content of anionic emulsifiers is usually <0.1% by weight, in particular <0.05% by weight and particularly preferably <0.01% by weight, based on the colorant-containing polymer PC (polymer matrix+encapsulated colorant C).

Homogeneous distribution of the organic colorant is understood as meaning that the organic colorant is distributed in molecularly disperse manner within the polymer matrix of the colorant-containing polymer, i.e. is present in monomolecularly dissolved form or dissolved in the form of bi- or polymolecular colorant aggregates.

The term colorant encompasses here and below chemical compounds or salts of chemical compounds, and charge transfer complexes of chemical compounds chosen from dyes, optical brighteners and UV absorbers. These compounds have an extended π-electron system having usually at least 8 π electrons. In the case of the dyes, these compounds have an absorption maximum in the wavelength range from 400 to 850 nm and thus give rise to a color impression for the human eye (conventional dyes) and in some cases also themselves emit light in the visible region (fluorescent dyes). Optical brighteners have one or more absorption maxima in the range from 250 to 400 nm and, upon irradiation with UV light, emit fluorescent radiation in the visible region. UV absorbers absorb light of wavelength <400 nm and convert it to heat radiation.

C₁-C₂₀-alkyl is here and below to be understood as meaning both linear and also branched alkyl having 1 to 20 carbon atoms. Examples thereof are methyl, ethyl, n-propyl, i-propyl, n-butyl, 2-butyl, isobutyl, tert-butyl, n-pentyl, isopentyl, neopentyl, tert-pentyl, hexyl, 2-methylpentyl, heptyl, 1-ethylpentyl, n-octyl, 2-ethylhexyl, isooctyl, nonyl, isononyl, decyl, isodecyl, undecyl, dodecyl, tridecyl, isotridecyl, tetradecyl, pentadecyl, hexadecyl, heptadecyl, octadecyl, nonadecyl, eicosyl.

C₅-C₁₀-cycloalkyl are to be understood as meaning aliphatic monocycles, such as cyclopentyl, cyclohexyl and cycloheptyl, and aliphatic polycycles, such as norbornyl, adamantyl or decahydronaphthyl.

It is essential for the invention that the colorant C can be distributed homogeneously in the polymeric matrix of the colorant-containing polymer. This is generally ensured if the organic colorant C, optionally in the form of a salt, has an at least limited solubility in the low molecular weight constituents which form the polymer matrix (monomer M). The organic colorant C preferably has the solubility which is greater than the intended use amount in the polymer. Suitable colorants C therefore have a solubility of >1% by weight, in particular >2% by weight, in particular >5% by weight and very particularly preferably >10% by weight in the monomers M.

Depending on the absorption strength of the colorant C, the colorant-containing polymer PC generally comprises at least 0.5% by weight, based on the weight of the polymer matrix, preferably 1 to 40% by weight and in particular 2 to 30% by weight, of at least one organic colorant C.

Examples of monomer-soluble, neutral dyes are the compounds which, according to the Colour Index, are referred to as disperse dyes and as solvent dyes, which are also referred to as dispersion dyes. A list of suitable dispersion dyes is given, for example, in Ullmann's Encyclopedia of Industrial Chemistry, 4^{th} Edition, Vol. 10, pp. 155-165 (see also Vol. 7, p. 585 et seq.—Anthraquinone dyes; Vol. 8, p. 244 et seq.—Azo dyes; Vol. 9, p. 313 et seq.—Quinophthalone dyes). Express reference is hereby made to this literature reference and the compounds mentioned therein.

Dispersion dyes and solvent dyes which are suitable according to the invention include a very wide variety of classes of dye with various chromophores, for example anthraquinone dyes, monoazo and disazo dyes, quinophthalones, methine and azamethine dyes, naphthalimide dyes, naphthoquinone dyes and nitro dyes. Examples of dispersion dyes suitable according to the invention are the dispersion dyes of the following Colour Index List:

C. I. Disperse Yellow 1, 2, 3, 4, 5, 6, 7, 8, 9, 10, 11, 11:1, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 179, 180, 181, 182, 183, 184, 184:1, 200, 201, 202, 203, 204, 205, 206, 207, 208, 209, 210, 211, 212, 213, 214, 215, 216, 217, 218, 219, 220, 221, 222, 223, 224, 225, 226, 227, 228.

C. I. Disperse Orange 1, 2, 3, 3:3, 4, 5, 6, 7, 8, 9, 10, 11, 12, 13, 14, 15, 16, 17, 18, 19, 20, 21, 22, 23, 24, 25, 25:1, 26, 27, 28, 29, 30, 31, 32, 33, 34, 35, 36, 37, 38, 39, 40, 41, 41:1, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 126, 127, 128, 129, 130, 131, 136, 137, 138, 139, 140, 141, 142, 143, 145, 146, 147, 148.

C. I. Disperse Red 1, 2, 3, 4, 5, 5:1, 6, 7, 8, 9, 10, 11, 12, 13, 14, 15, 16, 17, 18, 19, 20, 21, 22, 23, 24, 25, 26, 27, 28, 29, 30, 30:1, 31, 32, 33, 34, 35, 36, 38, 39, 40, 41, 43, 43:1, 46, 48, 50, 51, 52, 53, 54, 55, 55:1, 56, 58, 59, 60, 61, 63, 65, 66, 69, 70, 72, 73, 74, 75, 76, 77, 79, 80, 81, 82, 84, 85, 86, 86: 1, 87, 88, 89, 90, 91, 92, 93, 94, 96, 97, 98, 100, 102, 103, 104, 105, 106, 107, 108, 109, 110, 111, 112, 113, 115, 116, 117, 118, 120, 121, 122, 123, 125, 126, 127, 128, 129, 130, 131, 132, 133, 134, 135, 136, 137, 138, 139, 140, 141, 142, 143, 144, 145, 146, 147, 148, 149, 150, 151, 151:1, 152, 153, 154, 155, 156, 157, 158, 159, 160, 161, 162, 163, 164, 165, 166, 167, 167:1, 168, 169, 170, 171, 172, 173, 174, 175, 176, 177, 178, 179, 180, 181, 182, 183, 184, 185, 186, 187, 188, 189, 190, 190:1, 191, 191:1, 192, 193, 194, 195, 211, 223, 273, 274, 275, 276, 277, 278, 279, 280, 281, 302:1, 305, 306, 307, 308, 309, 310, 311, 312, 313, 314, 315, 316, 317, 318, 319, 320, 321, 322, 323, 324, 325, 326, 327, 328 329, 330, 331, 332, 333, 334, 335, 336, 338, 339, 340, 341, 342, 343, 344, 346, 347, 348, 349.

C. I. Disperse Violet 1, 2, 3, 4, 4:1, 5, 6, 7, 8, 9, 10, 11, 12, 13, 14, 15, 16, 17, 18, 19, 20, 21, 22, 23, 24, 25, 26, 27, 28, 29, 31, 33, 34, 35, 36, 37, 38, 39, 40, 41, 42, 43, 44, 45, 46, 47, 48, 49, 50, 51, 52, 53, 54, 55, 56, 57, 58, 59, 60, 81, 86, 87, 88, 89, 91, 92, 93, 94, 95, 96, 97.

C. I. Disperse Blue 1, 1:1, 2, 3, 3:1, 4, 5, 6, 7, 7:1, 8, 9, 10, 11, 12, 13, 13:1, 14, 15, 16, 17, 18, 19, 20, 21, 22, 23, 23:1, 24, 25, 26, 27, 28, 29, 30, 31, 32, 33, 34, 35, 36, 38, 39, 40, 42, 43, 44, 45, 47, 48, 49, 51, 52, 53, 54, 55, 56, 58, 60, 60:1, 61, 62, 63, 64, 64:1, 65, 66, 68, 70, 72, 73, 75, 76, 77, 79, 80, 81, 81:1, 82, 83, 84, 85, 86, 87, 88, 89, 90, 91, 92, 93, 94, 95, 96, 97, 98, 99, 100, 101, 102, 103, 104, 105, 106, 107, 108, 109, 111, 112, 113, 114, 115, 116, 117, 118, 119, 121, 122, 123, 124, 125, 126, 127, 128, 130, 131, 132, 133, 134, 136, 137, 138, 139, 140, 141, 142, 143, 144, 145, 146, 147, 148, 149, 150, 151, 152, 153, 154, 155, 156, 158, 159, 160, 161, 162, 163, 164, 165, 165:2, 166, 167, 168, 169, 170, 171, 172, 173, 174, 175, 195, 281, 282, 283, 283:1, 284, 285, 286, 287, 288, 289, 290, 291, 292, 293, 294, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 349.

C. I. Disperse Green 1, 2, 5, 6, 9.

C. I. Disperse Brown 1, 2, 3, 4, 4:1, 5, 7, 8, 9, 10, 11, 18, 19, 20, 21.

C. I. Disperse Black 1, 3, 4, 5, 6, 7, 8, 9, 10, 11, 12, 13, 14, 15, 20, 22, 24, 25, 26, 27, 28, 29, 29:1, 30, 31, 32, 33, 34, 36.

Examples of solvent dyes which are suitable according to the invention are the compounds of the following Colour Index List:

C. I. Solvent Yellow 2, 3, 7, 12, 13, 14, 16, 18, 19, 21, 25, 25:1, 27, 28, 29, 30, 33, 34, 36, 42, 43, 44, 47, 56, 62, 72, 73, 77, 79, 81, 82, 83, 83:1, 88, 89, 90, 93, 94, 96, 98, 104, 107, 114, 116, 117, 124, 130, 131, 133, 135, 141, 143, 144, 145, 146, 157, 160:1, 161, 162, 163, 167, 169, 172, 174, 175, 176, 179, 180, 181, 182, 183, 184, 185, 186, 187, 189, 190, 191.

C. I. Solvent Orange 1, 2, 3, 4, 5, 7, 11, 14, 20, 23, 25, 31A 40:1, 41, 45, 54, 56, 58, 60, 62, 63, 70, 75, 77, 80, 81, 86, 99, 102, 103, 105, 106, 107, 108, 109, 110, 111, 112, 113.

C. I. Solvent Red 1, 2, 3, 4, 8, 16, 17, 18, 19, 23, 24, 25, 26, 27, 30, 33, 35, 41, 43, 45, 48, 49, 52, 68, 69, 72, 73, 83:1, 84:1, 89, 90, 90:1, 91, 92, 106, 109, 111, 118, 119, 122, 124, 125, 127, 130, 132, 135, 141, 143, 145, 146, 149, 150, 151, 155, 160, 161, 164, 164:1, 165, 166, 168, 169, 172, 175, 179, 180, 181, 182, 195, 196, 197, 198, 207, 208, 210, 212, 214, 215, 218, 222, 223, 225, 227, 229, 230, 233, 234, 235, 236, 238, 239, 240, 241, 242, 243, 244, 245, 247, 248.

C. I. Solvent Violet 2, 8, 9, 11, 13, 14, 21, 21:1, 26, 31, 36, 37, 38, 45, 46, 47, 48, 49, 50, 51, 55, 56, 57, 58, 59, 60, 61.

C. I. Solvent Blue 2, 3, 4, 5, 7, 18, 25, 26, 35, 36, 37, 38, 43, 44, 45, 48, 51, 58, 59, 59:1, 63, 64, 67, 68, 69, 70, 78, 79, 83, 94, 97, 98, 99, 100, 101, 102, 104, 105, 111, 112, 122, 124, 128, 129, 132, 136, 137, 138, 139, 143.

C. I. Solvent Green 1, 3, 4, 5, 7, 28, 29, 32, 33, 34, 35.

C. I. Solvent Brown 1, 3, 4, 5, 12, 20, 22, 28, 38, 41, 42, 43, 44, 52, 53, 59, 60, 61, 62, 63.

C. I. Solvent Black 3, 5, 5:2, 7, 13, 22, 22:1, 26, 27, 28, 29, 34, 35, 43, 45, 46, 48, 49, 50.

Also suitable according to the invention are monomer-soluble derivatives of naphthalene, of anthracene, of perylene, of terylene, of quarterylene, and monomer-soluble diketopyrrolopyrrol dyes, perinone dyes, coumarin dyes, isoindoline and isoindolinone dyes, porphyrin dyes, phthalocyanine and naphthalocyanine dyes.

Suitable monomer-soluble coumarin dyes are described, for example, in U.S. Pat. No. 3,880,869 and DE-A 44 24 817, to which reference is hereby made in their entirety.

Suitable nonpolar perylene dyes are, for example, those described in U.S. Pat. No. 4,618,694, DE-A 24 51 782, U.S. Pat. No. 379,934, U.S. Pat. No. 4,446,324, EP-A 277 980, EP-A 657 436 or WO 96/22332. Further suitable nonpolar perylene dyes are given, for example, in EP-A 73 007. Reference is hereby made to said publications in their entirety. Examples of preferred perylene dyes are the 6,12-dicyanoperylene-1,7-dicarboxylic C₂-C₁₀-alkyl esters, the bis-(N-C₁-C₁₀-alkyl)perylenetetracarboxylic acid diimides and the corresponding N-(alkylphenyl) compounds, which are commercially available under the Lumogen®F trademarks (BASF Aktiengesellschaft, Germany), e.g. Lumogen®F Red 300, Lumogen®F Yellow 083 and Lumogen®F Orange 240.

Suitable naphthalene dyes include, inter alia, naphthalene-1,8-dicarboximides, which are substituted on the imide nitrogen by unsubstituted, linear or branched C₁-C₂₀-alkyl or aryl, and which can have C₁-C₆-alkoxy substituents in the 4- and/or the 5-position of the naphthalene ring.

Suitable anthracene dyes include, inter alia, 9,10-diphenylanthracene, 9,10-bisphenylethynylanthracene, 1,8-dichloro-9,10-bisphenylethynylanthracene. Examples of suitable anthracene dyes can be found, for example, in Ullmann's Encyclopedia of Industrial Chemistry, 5th Edition, Vol. A2, p. 402 et seq.

Suitable porphyrin dyes include, for example, tetraphenylporphyrin and octaethylporphyrin and zinc or nickel complexes thereof.

Examples of suitable phthalocyanin dyes are metallophthalocyanines, in particular copper phthalocyanines which, on the phenylene units of the chromophore, have solubilizing alkyl groups having, preferably, 4 to 20 carbon atoms, where the alkyl radicals may be bonded to the chromophore directly or via a functional group, for example via a sulfonamide group. Commercially available products are, for example, tetra-C₄-C₁₀-alkylphthalocyanine complexes, such as tetra-tert-butylcopper phthalocyanine or tetra-n-octylcopper phthalocyanine, and sulfonamides of mono- or polysulfonated metallophthalocyanines with C₁₀-C₂₀-alkylamines, e.g. the tetrasulfonamide of the tetrasulfonated copper phthalocyanine with stearylamine.

Also suitable as colorant C are ionic dyes. These dyes are not generally soluble as such in the polymer matrix, but can be converted into an oil-soluble form, i.e. a form soluble in the polymer matrix (=colorant C), by derivatization in accordance with a known method. In the case of customary cationic dyes, it is possible, for example, to exchange the anions for those anions which have long-chain alkyl radicals. Anions having long-chain alkyl radicals include, for example, the anions of long-chain carboxylic acids having 8 to 22 carbon atoms, mono- and dialkyl phosphates having 4 to 22 carbon atoms per alkyl radical, alkylsulfonates having 8 to 22 carbon atoms, e.g. dodecylsulfonate. Accordingly, dyes with basic groups, which are usually present in protonated form in the aqueous phase, can be reacted with the acids of the abovementioned anions to give oil-soluble salts of the dyes. Analogously, dyes containing acidic functional groups or containing anionic groups, e.g. sulfate or carboxylate groups, can be converted into a monomer-soluble form using long-chain amines or ammonium salts which have at least one long-chain organic radical. Suitable long-chain carboxylic acids, or salts thereof are derived from fatty acids, such as capric acid, palmitic acid, stearic acid, oleic acid, linoleic acid and linolenic acid. Suitable amines are, for example, primary, linear or branched-chain alkylamines having 8 to 22 carbon atoms in the alkyl radical.

These dyes are usually dyes of the abovementioned classes of dyes, for example mono- or bisazo dyes, which in each case have at least one sulfonic acid group, triarylmethane dyes which carry sulfonic acid groups, copper phthalocyaninesulfonic acid, sulfonic acid-group-containing quinoline dyes or stilbene dyes. The following Colour Index numbers may be mentioned by way of example:

Direct Yellow 4, 5, 11, 50, 127, 137, 147, 153;

Acid Orange 7, 8;

Direct Orange 15, 34, 102;

Direct Red 81, 239, 252-255;

Direct Violet 9, 51;

Acid Blue 9, 86;

Direct Blue 199, 218, 267, 273, 279, 281;

Acid Black 194, 208, 210, 221;

Direct Black 19, 161, 170 and 171.

Cationic or basic dyes include, for example, azo and bisazo dyes having amino groups or ammonium groups, triarylmethane dyes, or amine dyes, methine and azamethine dyes, for example Basic Red 1, Basic Red 14, Basic Blue 7, Basic Blue 11, Basic Blue 26, Basic Violet 1, Basic Violet 4, Basic Violet 10 etc. The monomer-soluble colorants C also include complexes of basic and acidic dyes or complexes of anionic and cationic dyes, for example the complex of chrysoidine base and metanil yellow acid.

According to the invention, the colorants C also include optical brighteners. Suitable optical brighteners are, for example, compounds from the classes of bisstyrylbenzenes, stilbenes, benzoxazoles, coumarins, pyrenes and naphthalenes. It is possible to use the abovementioned brighteners alone or else as mixtures with one another.

The abovementioned optical brighteners are usually commercially available products known per se. They are described, for example, in Ullmann's Encyclopedia of Industrial Chemistry, 5th edition, Volume A18, pp. 156-161, or can be obtained by the methods discussed therein.

The organic colorants C also include compounds which absorb UV rays (UV absorbers) which deactivate the absorbed radiation in a nonradiative manner. Such compounds are frequently used in sunscreens. UV absorbers include derivatives of p-aminobenzoic acid, in particular esters thereof; salicylates, substituted cinnamic esters (cinnamates), such as octyl p-methoxycinnamate, isopentyl 4-methoxycinnamate, benzophenones, such as 4-methoxy-2-hydroxybenzophenonesulfonic acid sodium salt, salicylates, such as 4-isopropylbenzyl salicylate, 4-aminobenzoic acid and its derivatives, such as ethoxylated ethyl 4-aminobenzoate, 2-ethylhexyl 4,4-dimethylaminobenzoate, esters of 4,4-diphenylbutadiene-1,1-dicarboxylic acid, e.g. bis(2-ethylhexyl) esters thereof,

2-phenylbenzimidazole-4-sulfonic acid and salts thereof, urocanic acid, salts thereof and esters thereof, benzoxazoles, benzotriazoles such as 2-(2H-benzotriazol-2-yl)-4-methyl-6-(2-methyl-3-((1,1,3,3-tetramethyl-1-(trimethylsilyloxy)disiloxanyl)propyl)phenol, benzylidenecamphor and its derivatives, as are given, for example, in DE-A 3 836 630, e.g. 3-benzylidenecamphor, 3-(4′-methylbenzylidene)-d-l-camphor, also a-(2-oxoborn-3-ylidene)toluene-4-sulfonic acid and its salts, N,N,N-trimethyl-4-(2-oxoborn-3-ylidenemethyl)anilinium methosulfate, dibenzoylmethanes such as

4-tert-butyl-4′-methoxydibenzoylmethane, 2,4,6-triaryltriazine compounds such as

2,4,6-tris-{N-[4-(2-ethylhex-1-yl)oxycarbonylphenyl]amino}-1,3,5-triazine,

4,4′-((6-(((tert-butyl)aminocarbonyl)phenylamino)-1,3,5-triazin-2,4-diyl)imino)bis(benzoic acid 2-ethylhexyl ester), ethyl and 2-ethylhexyl 2-cyano-3,3-diphenylacrylate, and

2-phenylbenzimidazole-5-sulfonic acid and its salts. Further UV absorbers suitable according to the invention are given in Cosmetic legislation, Vol.1, Cosmetic Products, European Commission 1999, pp. 64-66, to which reference is hereby made.

The nonionic surface-active compounds NS present in the colorant-containing polymer dispersions according to the invention naturally have at least one polar, hydrophilic molecular moiety, e.g. a molecular moiety having OH groups, e.g. a sorbitan, pentaerythritol or glyceryl radical, preferably a poly-C₂-C₃-alkylene ether group having at least 2 and up to 300 alkylene oxide repeat units and at least one lipophilic molecular moiety, preferably an aliphatic or alicyclic hydrocarbon radical which has at least 6 carbon atoms. Such compounds are known from the prior art as nonionic emulsifiers or surfactants or nonionic detergents, for example, from Ullmann's Encyclopedia of Industrial Chemistry, Vol. A9, 5^{th} ed., p.313 et seq. The nonionic surface-active compound preferably has at least one alkyl group having 6 to 32 carbon atoms and at least one oligoether group of the formula —[CH₂CH₂O]ₙ—H, in which n is at least 2 and is preferably in the range from 3 to 250. In this connection, it must be taken into consideration that the value n is an average value which is also referred to below as degree of ethoxylation.

Examples of compounds NS are

ethoxylates of linear or branched alkanols having 8 to 36, and in particular 10 to 22, carbon atoms and a degree of ethoxylation of from 3 to 250, in particular 3 to 50, e.g. ethoxylated C_{13/15}-oxo alcohol having 3 to 20 ethylene oxide units, C_{12/14}-fatty alcohol having 3 to 20 ethylene oxide units and polyethoxylated cetyl alcohol.

ethoxylates of mono-, di- and tri-C₄-C₁₂-alkylphenols having a degree of ethoxylation of from 3 to 50.

esters of C₈-C₃₂-fatty acids, such as stearic acid, palmitic acid, coconut fatty acid, tallow fatty acid, lauric acid or behenic acid with oligo- or polyethylene oxide which has, for example, a degree of oligomerization of from 2 to 100,

ethoxylates of C₈-C₂₂-mono- and dialkylamines, such as octylamine, stearylamine, di-n-octylamine and di-n-stearylamine having degrees of ethoxylation in the range from 3 to 50;

ethoxylates of amides of the abovementioned C₈-C₃₂-fatty acids having degrees of ethoxylation in the range from 3 to 50;

fatty acid esters of ethoxylated glycerol or ethoxylated sorbitan, e.g. polyethoxylates of hydrogenated castor oil, ethoxylated sorbitan monolaurate (degree of ethoxylation from 3 to 50, e.g. 20).

The compounds NS are present in the polymer dispersions according to the invention in an amount of, preferably, 0.2 to 10 and in particular from 0.3 to 5% by weight, based on the polymer matrix of the polymer PC.

For the stabilization of the polymer particles, the polymer dispersion according to the invention comprises, in an amount of from 1 to 50% by weight, preferably 2 to 30% by weight, and in particular 5 to 25% by weight, based on the polymer matrix, at least one amphiphilic polymer PA which has 0.5 to 10 mol/kg and preferably 1 to 8 mol/kg of polymer PA of anionic functional groups.

Anionic groups are naturally derived from acid groups, from which they arise as a result of deprotonation. Anionic groups include, for example, carboxylate, sulfonate, sulfate, phosphonate and phosphate groups. Preferred polymers PA have carboxylate groups as anionic groups. Suitable counterions are either alkali metal ions, e.g. sodium or potassium ions, or ammonium ions. In view of the use of the polymer dispersions according to the invention in cosmetic compositions, counterions of interest are, in particular, NH₄⁺ and the ammonium ions of skin-compatible organic amines, in particular of amino alcohols, such as

2-amino-2-methylpropan-1-ol, monoethanolamine, diethanolamine, triethanolamine, triisopropanolamine, tri[(2-hydroxy)-1-propyl]amine, 2-amino-2-methylpropane-1,3-diol, 2-amino-2-(hydroxymethyl)propane-1,3-diol and of amino acids, such as lysine.

Of course, it is not necessary for all of the acid groups in the polymer PA to be present in neutralized form. A degree of neutralization of 50% of all acid groups present in the polymer usually suffices. In particular, the degree of neutralization is 80 to 100%.

In addition to the polar anionic groups, the amphiphilic polymer naturally also has hydrophilic atomic groups, preferably in the form of C₁-C₃₂-alkyl groups, which may be bonded to the backbone of the polymer PA directly or via an intermediate member, for example via an oxygen atom, a polyether group, a carbonyl, carbonyloxy or carbonylamino group.

In the polymer PA, some, preferably not more than 50 mol %, e.g. 1 to 50 mol %, in particular 5 to 25 mol %, of the carboxyl groups can be esterified by a surface-active compound having oligoether group of the formula —[CH₂CH₂O]ₙ—H, in which n is at least 2, e.g. with one of the compounds NS described above.

In contrast to the anionic emulsifiers, the weight-average molecular weight of the polymers PA is usually above 1000 daltons and in particular above 2000 daltons. The weight-average molecular weight will preferably not exceed a value of 100000 daltons, in particular of 70000 daltons.

Preference is given to polymers PA which have a carbon polymer chain and are thus constructed from ethylenically unsaturated monomers M′.

In preferred polymers PA, the monomers M′ comprise:

5 to 70 mol %, in particular 10 to 60 mol %, of at least one monomer A, chosen from monoethlyenically unsaturated mono- and dicarboxylic acids having 3 to 8 carbon atoms;

30 to 95 mol %, in particular 40 to 90 mol %, of at least one monomer B which is insoluble or has limited solubility in water and optionally

up to 30 mol %, preferably up to 20 mol %, of a monomer C which is different from the monomers A and B, in each case based on the sum of the monomers A, B and C.

Examples of monomers A are acrylic acid, methacrylic acid, crotonic acid, vinyl acetic acid, 2-ethylacrylic acid, 2-acryloxyacetic acid, 2-acrylamidoacetic acid, maleic acid, mono-C₁-C₄-alkyl maleates, such as monomethyl and monobutyl maleates, fumaric acid, mono-C₁-C₄-alkyl fumarates, such as monomethyl and monobutyl fumarates, itaconic acid and 2-methylmaleic acid.

Monomers B with limited solubility in water are those which have a solubility in water of up to 80 g/l (at 25° C. and 1 bar). They determine the hydrophobic character of the polymers PA. Such monomers usually have at least one C₁-C₂₀-alkyl group. Examples of suitable monomers B are:

vinyl and allyl esters of aliphatic monocarboxylic acids having 2 to 20 carbon atoms, such as vinyl acetate, vinyl propionate, vinyl pivalate, vinyl versatate, vinyl laurate and vinyl stearate;

C₁-C₂₀-alkyl and C₅-C₁₀-cycloalkyl esters of the above-mentioned ethylenically unsaturated mono- and dicarboxylic acids, in particular of acrylic acid and of methacrylic acid. Preferred esters are methyl methacrylate, ethyl methacrylate, n-butyl methacrylate, tert-butyl methacrylate, isobutyl methacrylate, n-hexyl methacrylate, cyclohexyl methacrylate, 2-ethylhexyl methacrylate, methyl acrylate, ethyl acrylate, n-butyl acrylate, tert-butyl acrylate, isobutyl acrylate, cyclohexyl acrylate and 2-ethylhexyl acrylate;

mono- and di-C₁-C₂₀-alkylamides of the abovementioned ethylenically unsaturated mono- and dicarboxylic acids, in particular of acrylic acid and of methacrylic acid, e.g. N-tert-butylacrylamide and N-tert-butylmethacrylamide;

C₃-C₂₀-olefins, such as propene, 1-butene, isobutene, 2-methylbutene, 1-pentene, 2-methylpentene, 1-hexene, 2-methylhexene 1-octene, isooctene, 2,4,4-trimethylpentene (diisobutene).

vinylaromatic monomers, such as styrene, α-methylstyrene, vinyltoluene and p-tert-butylstyrene.

Suitable as monomers C are, preferably, monoethylenically unsaturated monomers. Particularly suitable are neutral monomers C which have a solubility in water above 80 g/l (at 25° C. and 1 bar). Examples of such monomers are the amides of the above-mentioned ethylenically unsaturated monocarboxylic acids, such as acrylamide and methacrylamide, N-vinyllactams such as N-vinylpyrrolidone and N-vinylcaprolactam, hydroxyalkyl esters of the abovementioned monoethylenically unsaturated carboxylic acids, such as hydroxyethyl acrylate, hydroxypropyl acrylate, hydroxybutyl acrylate, hydroxyethyl methacrylate, hydroxypropyl methacrylate, hydroxybutyl methacrylate and the esters of acrylic acid or of methacrylic acid with oligoalkylene oxides, such as oligoethylene oxide or oligopropylene oxide having degrees of oligomerization in the range from 2 to 200.

The term copolymer used here and below is not restricted to copolymers of two different monomers, but also includes polymers constructed from three or more different monomers.

Examples of preferred classes of polymers PA are:

copolymers of maleic acid as monomer A and C₃-C₂₀-olefins as monomer B in which 1 to 50 mol % and preferably 5 to 30 mol % of the carboxyl groups may be esterified by a surface-active compound containing oligoether group of the formula —[CH₂CH₂O]ₙ—H, in which n is at least 2. Such polymers are known, for example, from EP-A 116 930 and EP-A 367 049, to which reference is hereby made. Of these, mention may be made in particular of copolymers of methacrylic acid and diisobutene in the molar ratio 1:1, in which 1 to 50 mol % and in particular 5 to 30 mol % of the carboxylic acid groups have been esterified with an ethoxylated C₁₀-C₂₂-alkanol which has a degree of ethoxylation of from 3 to 50;

copolymers of monoethylenically unsaturated C₃-C₈-monocarboxylic acids as monomers A with vinyl esters of aliphatic C₂-C₂₀-carboxylic acids as monomers B, e.g. copolymers of vinylacetate with crotonic acid in the molar ratio 0.5:9.5 to 2:8;

copolymers of monomers A chosen from the C₁-C₂₀-alkyl esters of monoethylenically unsaturated monocarboxylic acids and the N-C₁-C₂₀-alkyl- and N,N-(di-C₁-C₂₀-alkyl)amides of monoethylenically unsaturated C₃-C₈-monocarboxylic acids. Examples which may be mentioned here are terpolymers of N-tert-butylacrylamide, ethyl acrylate and acrylic acid or of tert-butyl acrylate, ethyl acrylate and methacrylic acid having acid numbers in the range from 60 to 200 mg of KOH/g of polymer; corresponding to a carboxyl group content of from 1 to 3.5 mol/kg.

The colorant-containing polymer particles PC comprise a polymer matrix constructed according to the invention from ethylenically unsaturated monomers M. The weight-average particle size (particle diameter) of the polymer particles is preferably in the range from 50 to 500 nm and in particular in the range from 100 to 400 nm. The average particle size corresponds to the volume average which can be determined in a known manner by quasi elastic light scattering of a dilute aqueous dispersion of the polymer particles (calculated by unimodal analysis of the autocorrelation function). The measurements are usually made on 0.1% strength by weight samples under standard conditions (1 bar, 25° C.). The measurement can be made, for example, using a Coulter N4 Plus Particle Analyzer from Coulter Scientific Instruments.

The polymeric matrix usually comprises at least one hydrophobic monomer M1 having a solubility in water in the range from 0.01 g/l to 80 g/l, in particular 0.1 to 50 g/l (at 25° C. and 1 bar). The monomers A usually constitute at least 70% by weight and in particular at least 80% by weight, e.g. 70 to 99.9% by weight and in particular 80 to 99% by weight, based on the total weight of the monomers M.

The monomers M1 are preferably chosen from:

C₁-C₂₀-alkyl and C₅-C₁₀-cycloalkyl esters of the abovementioned ethylenically unsaturated mono- and dicarboxylic acids, in particular of acrylic acid and of methacrylic acid. Preferred esters are methyl methacrylate, ethyl methacrylate, n-butyl methacrylate, tert-butyl methacrylate, isobutyl methacrylate, n-hexyl methacrylate, cyclohexyl methacrylate, 2-ethylhexyl methacrylate, methyl acrylate, ethyl acrylate, n-butyl acrylate, tert-butyl acrylate, isobutyl acrylate, cyclohexyl acrylate and 2-ethylhexyl acrylate;

vinyl esters of C₁-C₈-monocarboxylic acids. Examples of vinyl esters are vinyl acetate, vinyl propionate, vinyl butyrate and vinyl hexanoate;

vinyl aromatic monomers as mentioned above, in particular styrene;

C₂-C₆-olefins, such as ethylene, propene, 1-butene, 2-butene and isobutene.

It has proven advantageous if the monomers M also comprise, as well as the monomers M1, crosslinking monomers M2 which have at least two nonconjugated ethylenically unsaturated double bonds. By virtue of the monomers M2, better incorporation of the colorant into the polymer matrix is achieved. The monomers M2 usually constitute 0.1 to 30% by weight, preferably 0.5 to 20% by weight, in particular 1 to 10% by weight, of the monomers M.

Suitable monomers M2 comprise, for example, the vinyl, allyl and methallyl esters of the above-mentioned ethylenically unsaturated carboxylic acids and likewise the esters of these acids with tricyclodecenyl alcohol, in particular the esters of methacrylic acid and of acrylic acid, the esters of the above-mentioned ethylenically unsaturated carboxylic acids with polyhydric alcohols, such as ethylene glycol diacrylate, ethylene glycol dimethacrylate, butanediol diacrylate, butanediol dimethacrylate, hexanediol diacrylate, hexanediol dimethacrylate, triethylene glycol diacrylate, triethylene glycol trimethacrylate, tris(hydroxymethyl)ethane triacrylate and trimethacrylate, pentaerythritol triacrylate and trimethacrylate, and also the allyl and methallyl esters of polyfunctional carboxylic acids, such as diallyl maleate, diallyl fumarate and diallyl phthalate. Typical monomers B1 are also compounds such as divinylbenzene, divinylurea, diallylurea, triallyl cyanurate, N,N′-divinyl- and N,N′-diallylimidazolidin-2-one, and methylenebisacrylamide and methylenebismethacrylamide.

With regard to the preparability of the colorant-containing aqueous polymer dispersions of the polymers PC according to the invention, it has also proven advantageous if particularly hydrophobic monomers (monomer M3) with a solubility in water of <0.01 g/l (at 25° C. and 1 bar) are present during the polymerization of the monomers M. Monomer M3 are, if desired, used in an amount of from 0.1 to 20% by weight, in particular in an amount of from 1 to 10% by weight, based on the monomers M.

Examples of monomers M3 which have a low solubility in water as required above are 2- and 4-n-butylstyrene, p-tert-butylstyrene, esters of α,β-monoethylenically unsaturated carboxylic acids having 3 to 6 carbon atoms and alkanols having ≧12 carbon atoms (usually up to 30 carbon atoms), such as, for example, lauryl acrylate and stearyl acrylate. The monomers M3 also include esters of vinyl alcohol or allyl alcohol and alkanecarboxylic acids having >9 carbon atoms (usually up to 30 carbon atoms), such as, for example, vinyl nonanoate, vinyl decanoate, vinyl laurate and vinyl stearate, and commercially available monomers VEOVA® 9-11 (VEOVA X is a tradename of Shell and stands for vinyl esters of carboxylic acids, which are also referred to as Versatic® X acids). The monomers M3 include macromonomers, such as oligopropene acrylate (very generally, macromonomers are polymeric or oligomeric compounds which have at least one, in most cases, terminal, ethylenically unsaturated double bond; their relative number-average molecular weight should preferably be not more than 100000 for applicability as least water-soluble monomer M3; usually this relative number-average molecular weight is 1000 to 50000 or 2000 to 50000; macromonomers are known to the person skilled in the art; their preparation is described, for example, in Makromol. Chem. 223 (1994) p. 29 to 46). Very generally, suitable as least water-soluble monomers M3 are all those whose molecular solubility at 25° C. and 1 atm in water is equal to or less than the corresponding solubility of lauryl acrylate. Such monomers M3 are, for example, also the methacryloyl polybutyl acrylate AB-6 and the methacryloyl polystyrene A5-6 from Toa Gosei Kagaku KK (JP), both of which have a number-average relative molecular weight of 6000. However, Polyol 130 and Polyol 110 from Hüls AG (stereospecific, low-viscosity polybutadiene (75% 1,4-cis, 24% 1,4-trans, 1% vinyl), whose dynamic viscosity at 20° C. is 3000 mPas) are also monomers M3 which can be used as macromonomers with low solubility in water. Instead of the polymers M3, it is also possible to use nonpolymerizable compounds with a solubility in water of <0.01 g/l in the preparation of the colorant-containing aqueous polymer dispersions according to the invention.

The monomers M may also comprise, in an amount of up to 30% by weight, preferably not more than 20% by weight and in particular not more than 10% by weight, monomers M4 which are different from the above-mentioned monomers M1, M2 and M3. These include monomers whose homopolymers have increased solubility in water (i.e. >80 g/l at 25° C.). Such monomers M4 serve as modifying monomers. The monomers M4 include both monoethylenically unsaturated monomers having at least one acid group, e.g. a COOH—, SO₃H— or PO₃H₂— group, which may also be present in salt form (referred to below as anionic monomers) and monoethylenically unsaturated, neutral monomers.

Examples of monoethylenically unsaturated anionic monomers D (monomers DA) are the abovementioned monoethylenically unsaturated mono- and dicarboxylic acids, in particular acrylic acid and methacrylic acid, monoethylenically unsaturated sulfonic acids and phosphonic acids, e.g. vinylsulfonic acid, allylsulfonic acid, methallylsulfonic acid, styrenesulfonic acid, vinylnaphthalenesulfonic acid and (meth)acrylamido-2-methylpropanesulfonic acid, and also vinylphosphonic acid, allylphosphonic acid, methallylphosphonic acid, styrenephosphonic acid, and (meth)acrylamido-2-methylpropanephosphonic acid, and water-soluble salts thereof, e.g. alkali metal salts thereof or ammonium salts thereof, in particular sodium salts thereof. Examples of neutral monomers M4 are, in particular, the amides of monoethylenically unsaturated mono- and dicarboxylic acids, such as acrylamide, methacrylamide, also N-vinyllactams having 3 to 8 carbon atoms, such as N-vinylpyrrolidone and N-vinylcaprolactam. The monomers M4 also include acrylonitrile and methacrylonitrile, the use of which frequently leads to better solubility of the colorant in the monomers M and thus to better distribution of the colorant within the polymeric matrix. Acrylonitrile and methacrylonitrile are, if desired, often used in amounts up to 30% by weight, e.g. in amounts of from 0.5 to 30% by weight, based on the total weight of the monomers M, in the preparation of the colorant-containing polymers PC.

Of the abovementioned polymers, preference is given for the use according to the invention in particular to those colorant-containing polymers PC in which 50% by weight of the monomers M1 are chosen from those monomers whose homopolymers have a glass transition temperature of >40° C. (monomers M1a), and have less than 50% by weight, in particular less than 30% by weight and particularly preferably less than 10% by weight, of those monomers M1b whose homopolymers have a glass transition temperature of <40° C. Examples of particularly preferred monomers M1a are methyl acrylate, methyl methacrylate, ethyl methacrylate, tert-butyl acrylate, vinyl acetate, styrene, vinyltoluene and methylacrylonitrile.

In a very particularly preferred embodiment of the colorant-containing polymers according to the invention, the polymeric matrix is constructed from:

80 to 99% by weight of monomers M1, in particular monomers M1a, particularly preferably of methyl acrylate, styrene, methyl methacrylate or mixtures thereof, where up to 30% by weight of the monomers M1 can be replaced by acrylonitrile,

1 to 20% by weight, in particular 2 to 10% by weight, of monomers M2, e.g. divinylbenzene or 1,4-butanediol diacrylate,

0 to 20% by weight, e.g. 1 to 20% by weight, of monomers M3, e.g. lauryl acrylate or stearyl acrylate, and

0 to 20% by weight, e.g. 1 to 20% by weight, of monomers M4, e.g. acrylic acid, methacrylic acid, acrylamide, methacrylamide, acrylamido-2-methylpropanesulfonate sodium salt.

The preparation of the aqueous dispersions of colorant-containing polymers PC according to the invention is carried out by free-radical aqueous emulsion polymerization of an oil-in-water emulsion of the monomers M whose monomer droplets (=emulsion droplets) comprise the colorant in dissolved form and which, in contrast to conventional oil-in-water emulsions, have a droplet diameter of <1000 nm. Such finely divided monomer emulsions are also referred to as “mini-Emulsions” (cf. P. L. Tang, E. D. Sudol, C. A. Silebi and M. S. El-Aasser in Journal of Applied Polymer Science, Vol. 43, pp. 1059-1066 [1991]). The droplets preferably have an average diameter d_{z} of ≦500 nm and in particular ≦400 nm. D_{z} is usually at least 40 nm and preferably at least 100 nm. The droplet size of the oil-in-water emulsion of the monomers is determined in a similar way to the particle size of the polymer particles of the polymer PC by quasi elastic, dynamic light scattering. The droplets in the colorant-containing monomer emulsion preferably have a largely uniform size, i.e. the quotient (d₉₀-d₁₀)/d₅₀ has a value of ≦1, preferably ≦0.5, in particular ≦0.25. Here, dₙ stands for the particle diameter below which n % by weight of the emulsion droplets fall.

For the preparation of the colorant-containing monomer miniemulsion, the colorant C is firstly dissolved in the monomers M to be polymerized. The resulting colorant solution is then converted into an oil-in-water emulsion by customary methods, for example by stirring or dispersing it into an aqueous solution of a surface-active substance. The surface-active substance is chosen according to the invention from the above-defined nonionic compounds NS and the amphiphilic polymer PA. For the emulsification, either some or all of the amount of compound NS and amphiphilic polymer PA required for the preparation of the polymer dispersion according to the invention is used. For example, the emulsification can be carried out in the presence of compound NS as the sole surface-active substance and then, after the emulsification, the amphiphilic polymer PA can be added, and vice versa.

The resulting aqueous emulsions generally have average droplet sizes d_{z} above 1000 nm. These conventional “macroemulsions” are then converted into monomer emulsions with droplet sizes of ≦1000 and preferably ≦500 nm by means of homogenization. The polymerization of monomer emulsions with droplet sizes of ≦500 nm leads to particularly high-value colorant-containing polymers PC.

The homogenization is preferably carried out using ultrasound (e.g. Branson Sonifier II 450). For the homogenization using ultrasound, devices described in GB 22 50 930 A and U.S. Pat. No. 5,108,654 are suitable. The use of ultrasound has proven particularly successful for the preparation of the colorant-containing miniemulsions and generally leads to particularly high-value colorant-containing polymers PC.

The polymerization of the miniemulsion is then carried out in the presence of a polymerization initiator which triggers the free-radical polymerization of the monomers M, where, according to the invention, the polymerization takes place in the presence of 0.1 to 20% by weight of at least one nonionic surface-active compound NS and 1 to 50% by weight, in each case based on the monomers M, of at least one amphiphilic polymer PA.

In this connection, the procedure may involve initially introducing the colorant-containing emulsion, preferably in the form of a miniemulsion, into the reactor, and adding the polymerization initiator thereto under polymerization conditions in one portion or in two or more portions or continuously according to its consumption. It is also possible to firstly add some or all of the polymerization initiator to the amount of emulsion and then heat to the polymerization temperature.

It is also possible to add some or all of the aqueous, colorant-containing monomer emulsion, preferably in the form of a miniemulsion, to the polymerization vessel under polymerization conditions according to the progress of the reaction. Here, preference is given to introducing the polymerization initiator into the polymerization vessel at least partially in parallel to the addition of the monomer emulsion.

Suitable free-radical polymerization initiators are, in principle, all those able to trigger a free-radical polymerization. These are peroxides, hydroperoxides and also azo compounds. The free-radical polymerization initiators may either be water-soluble or oil-soluble, i.e. soluble in the monomers.

Examples of water-soluble initiators are peroxodisulfuric acid and its ammonium and alkali metal salts, hydrogen peroxide and low molecular weight hydroperoxides, such as tert-butyl hydroperoxide, or salt-like azo compounds, e.g. 2,2′-azobis-2-amidinopropane dihydrohalide.

Examples of oil-soluble polymerization initiators are C₄-C₁₂-peroxocarboxylic acids and their esters, e.g. peroctoate and perbenzoate, such as tert-butyl peroctoate and tert-butyl perbenzoate, and diacyl peroxides, such as dibenzoyl peroxide.

The abovementioned water-soluble peroxidic polymerization initiators can also be combined with a reducing agent and optionally with a metal compound which is soluble in the aqueous medium (redox initiator systems). These are sufficiently known to the person skilled in the art. Reference is made to WO 99/40123 for further details.

The amount of initiator used is generally in the range from 0.1 to 10% by weight, preferably 0.2 to 8% by weight and frequently in the range from 0.3 to 5% by weight.

In the preparation of the aqueous dispersions of the colorant-containing polymers PC, the polymerization temperatures are primarily governed by the initiator system used in each case in a known manner. The polymerization temperatures are usually in the range from 0 to 95° C., preferably in the range from 30 to 90° C. If increased pressure is used, the polymerization temperature can also be up to 120° C. Polymerization is usually carried out at atmospheric pressure (1 atmosphere).

The process according to the invention produces the colorant-containing aqueous polymer dispersions according to the invention without noteworthy coagulation formation, without anionic emulsifiers being required to stabilize the polymer particles and the monomer droplets. The colorant-containing aqueous polymer dispersions according to the invention thus comprise, as surface-active substance, merely the above-described compounds NS and at least one amphiphilic polymer PA. In addition, the polymer dispersions according to the invention are characterized by good storage stability.

The present invention further relates to colorant-containing polymer powders obtainable by evaporating the volatile constituents of the aqueous colorant-containing polymer dispersion according to the invention. The evaporation of the volatile constituents is carried out analogously to the known processes of powder preparation from aqueous polymer dispersions.

Surprisingly, the evaporation of the volatile constituents is possible without the customarily required addition of spraying auxiliaries. In contrast to conventional polymer dispersions, the polymer dispersions according to the invention do not display any undesired irreversible aggregation of the polymer particles during evaporation of the volatile constituents. The polymer powders can therefore be readily redispersed again in an aqueous phase.

The evaporation of the volatile constituents is carried out, for example, by spray-drying the colorant-containing polymer dispersion according to the invention in a stream of warm air or by freeze-drying. Processes for the spray-drying and the freeze-drying of aqueous polymer dispersions are known in principle to the person skilled in the art.

In the case of spray-drying, the procedure may, for example, involve spraying the polymer dispersions to be dried in a customary drying tower in a stream of warm air. Here, the inlet temperature of the stream of warm air is in the range from 100 to 200° C., preferably 120 to 160° C., and the exit temperature of the stream of warm air is in the range from 30 to 90° C. and preferably 60 to 80° C. The spraying of the aqueous polymer dispersion in the stream of warm air can, for example, be carried out using single-component or multi-component nozzles or via a rotating disk. The polymer powders are usually separated off using cyclones or filter separators. The sprayed aqueous polymer dispersion and the stream of warm air are preferably introduced in parallel.

It is of course also possible to add drying auxiliaries to the polymer dispersions according to the invention prior to the drying process; these are also referred to as spraying auxiliaries. Suitable for this purpose are neutral and anionic, water-soluble polymers. These usually have a weight-average molecular weight MN in the range from 1000 to 100000, preferably 2000 to 100000.

Specific examples of neutral polymers are: polyvinyl alcohols (see e.g. EP-A-56 622, EP-A-680 993, DE-A-22 14 410 and DE-A-26 14 261), polyvinylpyrrolidones (see e.g. DE 22 38 903 and EP 576 844). Examples of anionic polymers are phenolsulfonic acid/formaldehyde condensates (see e.g. EP-A 407 889, WO 98/03576), naphthalenesulfonic acid formaldehyde condensates (see e.g. WO 98/03577), homopolymers and copolymers of 2-acrylamido-2-methylpropanesulfonic acid (see e.g. EP-A 629 650, EP-A 671 435 and DE-A 195 39 460), homo- and copolymers of ethylenically unsaturated carboxylic acids, such as, in particular, acrylic acid, methacrylic acid and maleic acid, with hydroxyalkyl esters (see e.g. JP 59 162 161).

The present invention further relates to cosmetic compositions which comprise at least one colorant-containing polymer PC in the form of an aqueous polymer dispersion of a colorant-containing polymer powder and the adjuvants customary for cosmetic compositions.

Examples of cosmetic compositions are compositions for treating the facial skin, in particular in the eye area, such as kohl pencils, eyeliner pencils, eyebrow pencils, eyeshadows, cream blusher, powder blusher, foundation, make-up, e.g. theater make-up, lipsticks; compositions for treating eyebrows and eyelashes, such as mascara and eyelash make-up; nail varnishes, both solvent-based and water-based; hair-treatment compositions, such as hairgels, e.g. wet gel, styling gel, hairsprays, hair mascara, styling mousse, hair foam, hair shampoo; also colored soaps and sunscreen compositions, e.g. sunblock creams and sunblock sticks. The latter are a preferred embodiment of the cosmetic compositions according to the invention and comprise, as colorant C, at least one of the abovementioned UV absorbers.

In the case of water-based cosmetic compositions, for example aqueous nail varnishes, mascara, foundations of the O/W type or foundations of the W/O type, in the case of decorative haircare compositions, such as wet gel, styling gel, hairspray, hair mascara or styling mousse, for the sake of simplicity, use is preferably made of aqueous dispersions of the colorant-containing polymers PC. By contrast, in the case of cosmetic compositions which consist exclusively of oils or fats, in particular those which have a solid form, e.g. pencils, such as kohl pencils, eyeliner pencils, eyebrow pencils, stick-shaped theater make-up, lipsticks and the like, and in the case of powder or fine powder cosmetic compositions, such as eyeshadows and cream blusher or loose powder blusher, use is made of a pulverulent colorant-containing polymer PC.

The amount of colorant-containing polymer PC in the cosmetic composition is primarily governed by the desired color impression which the decorative cosmetic composition is to have. Depending on the nature of the cosmetic composition and of the desired color impression, the content of colorant-containing polymer in the cosmetic composition is in the range from 0.1 to 50% by weight, based on the total weight of the cosmetic composition.

In the case of nail varnishes, 1 to 10% by weight, based on the nail varnish, of colorant-containing polymer PC is used, for example. In the case of mascara and eyelash make-up, 2 to 20% by weight, based on the cosmetic composition, of at least one colorant-containing polymer PC is used as color-imparting constituent. In cosmetic pencils, such as kohl pencils, eyeliner pencils, eyebrow pencils, eyeshadow pencils, 10 to 40% by weight of the colorant-containing polymer is generally used. In the case of eyeshadows, the content of polymer PC is usually higher still and can be as much as 50% by weight. In the case of cream blusher and loose powder blusher, the content of colorant-containing polymer PC is frequently in the range from 1 to 20% by weight, in particular in the range from 2 to 15% by weight. In the case of lipsticks, use is frequently made, depending on the desired color impression, of 2 to 40% by weight, in particular 5 to 30% by weight, of colorant-containing polymer, based on the total weight of the lipstick. In the case of hair gel and styling gel and in the case of hair spray, lower contents of colorant-containing polymer PC, e.g. 0.1 to 10% by weight, in particular 0.5 to 5% by weight, are generally used.

Of course it is additionally possible to also use other pigments of the prior art, it being possible for said pigments to partially replace the colorant-containing polymers PC or to supplement them to change the color impression. The amount of additional prior art pigments is generally in the range from 0.1 to 30% by weight in the cosmetic compositions according to the invention, based on the total weight of the cosmetic composition and is naturally governed by the type of cosmetic composition and the desired color impression.

The finely divided colorant-containing polymers PC used in accordance with the invention have, compared with the prior art pigments, firstly the advantage that they can be incorporated into the cosmetic compositions more readily since binding and digestion of the pigment is not required. This is true both for the aqueous polymer dispersions and for the powders of the polymer PC. The possible use of aqueous polymer dispersions facilitates in particular also the preparation of those formulations which have a high water content and a low or no fat content. In addition, the colorant-containing polymers PC have a higher color depth than comparable pigments of the prior art. In contrast to the pigments of the prior art, if the shade of the cosmetic composition is changed, it is not necessary to specifically adapt the other constituents to the new color-imparting constituent. Since it is possible to incorporate the most varied colorants into one type of polymer matrix, by matching the polymer matrix to the respective cosmetic composition once, it is possible to provide a broad pallet of colorants.

The cosmetic composition according to the invention can be in the form of a suspension or dispersion in solvents or fatty substances, in the form of an emulsion, such as, for example, a cream or milk, in the form of a pomade, gel or solid stick; it may be formulated as an aerosol or be in the form of a foam.

The cosmetic composition comprises the cosmetic adjuvants customary for the respective type of composition, such as thickeners, emollients, hydrating products, interface-active agents, preservatives, sequestering agents, antioxidants, antifoams, oils, waxes, lanolin, perfumes, propellants, colorants, vitamins or other ingredients customarily used in cosmetics.

Examples of customary cosmetic ingredients and numerous formulation examples of cosmetic compositions are given in K. Schrader, Grundlagen und Rezepturen der Kosmetika [Cosmetics principles and formulations], 2^{nd} edition, Hüthig-Buchverlag, Heidelberg, 1989.

If the composition is formulated as an aerosol, use is made of conventional propellants, such as alkanes, dinitrogen oxide and dimethyl ether.

Adjuvants which are in principle present in the cosmetic compositions of the invention include solvents, such as water, lower monoalcohols or polyols having 1 to 6 carbon atoms or mixtures thereof; the particularly preferred monoalcohols or polyols are ethanol, isopropanol, propylene glycol, glycerol and sorbitol; also present are fatty substances, such as mineral, animal, vegetable or synthetic oils or waxes, fatty acids, fatty acid esters, such as triglycerides of C₆-C₁₂-fatty acids, fatty alcohols, Vaseline, paraffin, lanolin, hydrogenated lanolin, acetylated lanolin and silicone oil.

Apart from comprising the polymers PC, emulsions in the form of a cream or a foundation comprise fatty alcohols, fatty acid esters and, in particular, fatty acid triglycerides, fatty acids, lanolin and derivatives thereof, natural or synthetic oils or waxes and emulsifiers in the presence of water.

The concentration of the emulsifier system is generally 4 to 35% by weight, based on the total weight of the emulsion; the fatty phase often constitutes between 10 and 90%, and the aqueous phase between 10 and 90%, based on the total weight of the emulsion. The emulsifiers are those customarily used in this type of emulsion. In particular, they are chosen from:

C₁₂-C₁₈-sorbitan fatty acid esters,

esters of hydroxystearic acid and C₁₂-C₃₀-fatty alcohols,

mono- and diesters of C₁₂-C₁₈-fatty acids and glycerol or polyglycerol,

condensates of ethylene oxide and propylene glycols,

oxypropylenated/oxyethylenated C₁₂-C₂₀-fatty alcohols,

polycyclic alcohols, such as sterols,

aliphatic alcohols with a high molecular weight, such as lanolin,

mixtures of oxypropylenated/polyglycerolated alcohols and magnesium isostearate,

succinic esters of polyoxyethylated or polyoxypropylenated fatty alcohols,

the lanolates and stearates of magnesium, calcium, lithium, zinc or aluminum, optionally as a mixture with hydrogenated lanolin, lanolin alcohol, or stearic acid or stearyl alcohol.

The fatty products which form the fatty phase of the emulsions include:

hydrocarbon oils, such as paraffin oil, purcellin oil, perhydrosqualene and solutions of microcrystralline waxes in these oils,

animal or vegetable oils, such as sweet almond oil, avocado oil, calophylum oil, lanolin and derivatives thereof, castor oil, horse oil, pig oil, sesame oil, olive oil, jojoba oil, karité oil, Hoplostethus oil,

mineral oils whose distillation start-point under atmospheric pressure is at about 250° C. and whose distillation end-point is at 410° C., such as, for example, Vaseline oil,

esters of saturated or unsaturated fatty acids, such as alkyl myristate, e.g. isopropyl, butyl or cetyl myristate, hexadecyl stearate, ethyl or isopropyl palmitate, octanoic or decanoic triglycerides and cetyl ricinoleate.

The fatty phase can also comprise silicone oils which are soluble in other oils, such as dimethylpolysiloxane, methylphenylpolysiloxane and the silicone glycol copolymer, fatty acids and fatty alcohols.

In order to favor the retention of oils, it is also possible to use waxes, such as, for example, carnauba wax, candelilla wax, beeswax, microcrystalline wax, ozokerite wax and Ca, Mg and Al oleates, myristates, linoleates and stearates.

The emulsions can also be in the form of a stick. In this case, the concentration of the water phase in the emulsion is generally 5 to 70% by weight, based on the total weight of the emulsion.

Fatty gels generally comprise an oil or a wax and a thickener, such as siliceous earth. The oily-alcoholic or aqueous-alcohol gels comprise one or more lower alcohols and polyols, such as ethanol, propylene glycol or glycerol, a thickener, such as siliceous earth, cellulose derivatives, polyacrylic acid derivatives and guar, carob and xanthan gum in the presence of oil or of water.

Solid sticks generally consist of fatty substances, such as natural or synthetic waxes and oils, fatty alcohols, fatty acid esters and lanolin.

Cosmetic compositions based on water frequently comprise gel formers, such as hydrocolloids and semisolid fats and waxes, e.g. guar gum, xanthan gum, tragacanth, alginates, starch, starch derivatives, gelatin, cellulose and cellulose derivatives, such as methylcellulose, sodium carboxymethylcellulose, hydroxypropylcellulose, hydroxyethylcellulose and polyacrylates.

The examples, given below, of colorant-containing polymer dispersions and colorant-containing powders, and the cosmetic formulation examples serve to illustrate the present invention in more detail:

I. General

1. Analysis

The polymer particle size (d_{z} value) was determined in the manner described above using a Coulter N4 Plus Particle Analyzer on 0.01% strength by weight samples of the dispersion.

The relative light transmission (LT value) for white light was determined on a 0.01% strength by weight sample at a path length of 2.5 cm against water.

The viscosity was determined in an Eprecht viscometer A-III at 23° C. for 20 min.

2. Colorants used:

UV absorber 1: n-octyl 4-methoxycinnamate

UV absorber 2: bis(2-ethylhexyl) 4,4-diphenylbutadiene-1,1-dicarboxylate,

UV absorber 3: 4-tert-butyl-4′-methoxydibenzoylmethane,

UV absorber 4: 2,4,6-tris-{N-[4-(2-ethylhex-1-yl)oxycarbonylphenyl]amino}-1,3,5-triazine,

UV absorber 5: 2-(ethylhexyl) 2-cyano-3,3-diphenylacrylate (octocrylene)

3. Nonionic surface-active agent:

NS1 polyethoxylated, hydrogenated castor oil (EMANON CH25 from KAO Corporation Japan).

4. Amphiphilic polymers PA used:

Polymer PA 1: sodium salt of a copolymer of maleic acid and diisobutene in the molar ratio 1:1, in which some of the carboxyl groups have been esterified with ethoxylated C_{13/15}-oxo alcohol (degree of ethoxylation 7), as aqueous solution (45% by weight). The polymer has a K value of 20 (determined in accordance with Fikentscher as 1% strength by weight solution in N,N-dimethylformamide), corresponding to a molecular weight of about 3000 and an acid number of 440 mg of KOH/g of polymer.

Polymer PA 2: salt of a copolymer of tert-butyl acrylate, ethyl acrylate and methacrylic acid in the molar ratio 59:11:30 with 2-amino-2-methylpropanol as aqueous solution (14% by weight). The polymer has a K value of 37 (determined in accordance with Fikentscher as 1% strength by weight solution in ethanol) and an acid number of from 140 to 160 mg of KOH/g of polymer.

Polymer PA 3: salt of a copolymer of vinyl acetate and crotonic acid in the molar ratio 9:1 with 2-amino-2-methylpropanol as aqueous solution (18% by weight). The polymer has a K value of 34 (determined in accordance with Fikentscher as 1% strength by weight solution in ethanol) and an acid number of 67.6 mg of KOH/g of polymer.

Polymer PA 4: salt of a copolymer of tert-butylacrylamide, ethyl acrylate and acrylic acid in the molar ratio 43.5:44.5:12 with 2-amino-2-methylpropanol as aqueous solution (12% by weight). The polymer has a K value of 40 (determined in accordance with Fikentscher as 1% strength by weight solution in ethanol) and an acid number of from 140 to 160 mg of KOH/g of polymer.

II. Preparation of aqueous dispersions of UV absorber-containing polymers (Examples 1 to 5):

1. Preparation of the miniemulsion (general procedure)

A reaction vessel fitted with a stirrer was charged with deionized water, 1.2 g of nonionic surface-active agent NS1 and the respective polymer PA (as aqueous solution). The total amount of water was about 500 ml. A solution of the respective UV absorber in the monomers to be polymerized (monomer/colorant solution) was added thereto over the course of 2 minutes. The use amounts and type of amphiphilic polymer PA and the constituents of this solution are given in Table 1. The composition of the monomer solution in all of the examples was as follows: 5 g of stearyl acrylate, 5 g of butanediol diacrylate, 95 g of methyl methacrylate and 20 g of UV absorber. The mixture was then stirred for a further 10 minutes. The resulting, conventional, colorant-containing monomer emulsions were then homogenized using ultrasound as follows to give an aqueous monomer microemulsion.

The ultrasound source was a Branson Sonifier II 450. The ultrasound treatment was carried out with stirring of the emulsion by sonication for 5 minutes at the setting duty-cycle 25%, output 10, and sonication for 10 minutes at the setting duty cycle 100% and output 10. The droplet size (volume average) in the monomer emulsion was below 400 nm.

2. Polymerization of the colorant-containing miniemulsion (general procedure)

The miniemulsion obtained as in 1. was introduced into a polymerization vessel and heated to 80° C. The initiator solution (1 g of sodium peroxodisulfate in 38 ml of water) was then added in one portion with stirring, the mixture was left to after-react for 3.5 h at 80 to 85° C., then cooled to 25° C. and filtered to determine the coagulant fraction using a 75 μm screen. This gave an approximately 20% strength by weight aqueous dispersion of the polymer PC. The proportion of coagulation was low in all cases (<5%, based on the feed materials).

The light transmission (LT value), the pH, the viscosity and the storage stability of the resulting polymer dispersions are given in Table 1. The average particle size of the polymer particles was below 400 nm as a consequence of the preparation.

TABLE 1
UV absorber-containing polymer dispersions

| | Polymer | | | | | | | |
|---|---|---|---|---|---|---|---|---|
| | PA | | UV | SC¹⁾ | LT²⁾ | | Viscosity | Storage |
| Ex. | type [g] | | Absorber | [% by wt.] | [%] | pH | [mPas] | stability³⁾ |
| 1 | 1 | 18 | UV1 | 20.2 | 98.3 | 6.8 | 3.7 | 3 |
| 2 | 1 | 9 | UV1 | 19.4 | 96.4 | 6.5 | 3.7 | 2 |
| 3 | 1 | 18 | UV3 | 20.5 | 97.5 | 6.7 | 3.7 | 2 |
| 4 | 1 | 18 | UV4 | 21.5 | 96.8 | 7.0 | 3.7 | 2 |
| 5 | 2 | 18 | UV1 | 20.7 | 59.2 | 7.8 | n.d. | 2 |

¹⁾SC = Solids content
²⁾LT value (light transmission)
³⁾Storage stability was assessed after storage for one month at room temperature by reference to coagulation on the following scale:
1: large amount of coagulate, low storage stability
2: small amount of coagulate, storage-stable
3: no coagulate, very good storage stability

In the same manner, the use of 18 g of polymer PA3 and of 18 g of polymer PA4 instead of PA1 led to storage-stable polymer dispersions.

Redispersibility:

All of the polymers PC can be freeze-dried to give powders by cooling in a dry ice-acetone bath by applying a vacuum. These powders can be redispersed in mineral oil, 1,3-butylene glycol and propylene glycol with vigorous stirring.

Formulations of polymers PC in cosmetic care compositions are described below by way of examples. All data are in grams. The quantitative data for the aqueous dispersions of the colorant-containing polymer PC are based on the polymer proportion.

Nail Varnish Formulations (Formulation 1 to 3)

Formulation 1:

| | |
|---|---|
| 26.3 | nitrocellulose |
| 4.9 | copolymer of polyoxyisobutylene and methyleneurea |
| 7.8 | copolymer of butyl acrylate and vinyl isobutyl ether, 50% |
| | by weight in ethyl acetate (Acronal ®700 L 50% BASF) |
| 4.9 | methoxypropyl acetate |
| 53.5 | butyl acetate |
| 2.6 | polymer PC as aqueous dispersion |

Formulation 2:

| | |
|---|---|
| 16.0 | nitrocellulose |
| 4.0 | toluenesulfonamide/formaldehyde resin |
| 5.0 | dibutyl phthalate |
| 10.0 | butyl acetate |
| 10.0 | ethyl acetate |
| 10.0 | ethanol |
| 40.0 | toluene |
| 5.0 | polymer PC as aqueous dispersion |

The constituents, with the exception of the aqueous dispersion of PC, are dissolved. The colorant-containing polymer PC is then stirred in as aqueous dispersion, and the mixture is then homogenized.

Aqueous Nail Varnish: (Formulations 3 to 4)

Formulation 3:

| | |
|---|---|
| 27.2 | aqueous polyurethane dispersion |
| 13.8 | acrylic acid/styrene copolymer |
| 0.08 | polyacrylic acid thickener |
| 0.5 | butyl glycol acetate |
| 2.4 | polymer PC as aqueous dispersion |
| | water ad 100 |

The polyurethane is initially introduced as a finely disperse aqueous dispersion. The acrylic acid/styrene copolymer is added as aqueous dispersion with stirring. The acrylate thickener is then added with stirring. The mixture is further stirred until the material is of high viscosity. Finally, the aqueous dispersion of PC is stirred in.

Formulation 4:

As Formulation 3, but using 0.4 g of Acid Blue 74 Aluminium Lake and 2.0 g of polymer PC as aqueous dispersion.

Mascara

Formulation 5:

| | |
|---|---|
| 14.0 | demin. water |
| 0.2 | antioxidant (Oxynex 2004 from E. Merck, Darmstadt) |
| 2.5 | polyoxyethylene/polyoxypropylene block copolymer |
| | (Poloxamer 407 from BASF Aktiengesellschaft) |
| 3.5 | polyvinylpyrrolidone |
| 11.0 | ethanol |
| 0.7 | triethanolamine |
| 0.52 | polyacrylic acid (CTFA: Carbomer) |
| 57.58 | demin. water |
| 10.0 | polymer PC as aqueous dispersion |

The polyacrylic acid is left to swell in water, and then the clearly dissolved residual constituents are incorporated to give a gel with stirring. The aqueous dispersion of PC is then incorporated.

Eyelash Make-Up

Formulation 6:

| | |
|---|---|
| 80.8 | castor oil |
| 6.0 | caprylic/capric triglyceride |
| 0.2 | antioxidant (Oxynex 2004 from E. Merck, Darmstadt) |
| 2.0 | trihydroxystearin |
| 0.3 | polyvinylpyrrolidone |
| 2.0 | sorbitan oleate |
| 8.7 | polymer PC as powder |

The fatty constituents are dissolved in one another. The polyvinylpyrrolidone is then stirred in. The pulverulent polymer PC is then mixed in.

Cream Mascara

Formulation 7:

| | |
|---|---|
| 75.0 | petroleum distillate |
| 8.3 | quaternium-18 hectorite |
| 2.5 | propylene carbonate |
| 11.5 | aqueous dispersion of PC |
| 1.0 | ultramarine |
| 1.7 | vinylpyrrolidone/vinyl acetate copolymer |

The components of the fatty phase are processsed by means of strong shear forces to give a gel. The aqueous dispersion of the colorant-containing polymer PC and the vinylpyrrolidone/vinyl acetate copolymer is then incorporated and homogenized.

Kohl Pencil-Cosmetic Pencil

Formulation 8:

| | |
|---|---|
| 34.3 | hydroxylated lanolin |
| 17.10 | hydrogenated cocoglyceride |
| 2.9 | lanolin |
| 28.6 | glyceryl stearate |
| 17.1 | polymer PC as powder |

The fatty components are melted at 80° C. The pulverulent polymer PC is then mixed in, optionally perfumed, and molded by casting or extrusion to give leads for cosmetic pencils.

Eyeliner Pencil

Eyeliner pencil
Formulation 9:

| | |
|---|---|
| 30.0 | cyclomethicone |
| 6.7 | lanolin oil |
| 8.0 | carnauba wax |
| 3.3 | beeswax |
| 22.7 | paraffin oil |
| 2.7 | cetyl alcohol |
| 20.0 | polymer PC as powder |
| 5.6 | Pigment Blue 15 |
| 1.0 | iron oxide pigment |

Eyebrow pencil
Formulation 10:

| | |
|---|---|
| 78.0 | Cutina LM (lipstick material from Henkel KGaA, Düsseldorf) |
| 12.0 | ozokerite |
| 9.0 | polymer PC as powder |
| 1.0 | iron oxide pigment |

Eyeshadows
Formulation 11

| | |
|---|---|
| 20 | talc |
| 10 | potato starch |
| 5 | magnesium stearate |
| 45 | polymer PC as powder |
| 5 | ultramarine (Sicomet Blue P 77007) |
| 15 | eyeshadow binder |

Eyeshadow binder

| | |
|---|---|
| 35 | lanolin |
| 30 | isopropyl stearate |
| 30 | paraffin oil |
| 3 | perfume oil |
| 1 | carnauba wax |
| 1 | propylparaben |

The eyeshadow constituents are mixed homogeneously, and the pulverulent polymer PC and the color pigment (ultramarine) are stirred in. The binder constituents are melted at 70° C. The eyeshadow constituents are sprayed together with the molten and well-mixed binder. The mixture is then compressed at a pressing force of 40 to 60 bar. This gives an eyeshadow powder with a soft feel on the skin and a unique color effect.

Formulation 12:

As previous formulation, but using 50 g of powder of PC instead of the ultramarine/PC mixture.

Eyeshadow in Stick Form

Formulation 13:

| | |
|---|---|
| 15.0 | triglyceride of a C₁₈₋₃₆-acid |
| 5.0 | glyceryl behenate |
| 35.0 | mineral oil |
| 15.0 | mineral oil (and) lanolin alcohol |
| 0.2 | perfume oil |
| 0.8 | polyvinylpyrrolidone |
| 1.5 | talc |
| 27.5 | polymer PC as powder |

The fatty components are melted at 80° C., and the pulverulent polymer PC is mixed in. The mixture is then perfumed and molded by casting or extrusion to give leads for cosmetic pencils.

Eyeshadow Pencil

Formulation 14:

| | |
|---|---|
| 6.0 | beeswax |
| 5.0 | carnauba wax |
| 10.0 | Candelilla wax |
| 34.0 | hexyl laurate |
| 20.0 | castor oil |
| 20.0 | polymer PC as powder |
| 4.0 | chromium oxide green pigment |
| 1.0 | perfume oil |

Eyeshadow pencils from the two above formulations can also be formulated with mixtures of color pigments and pulverulent polymers PC instead of the pure pulverulent polymer PF.

Cream Blusher (Formulations 15 and 16)

Formulation 15:

| | |
|---|---|
| 5.5 | candelilla wax |
| 8.5 | beeswax |
| 3.0 | cetyl palmitate |
| 8.5 | paraffin oil |
| 43.0 | cetearyl octanoate |
| 3.0 | hydrogenated coconut fatty acid glyceride |
| 11.0 | vaseline |
| 14.5 | talc |
| 3.0 | polymer PC as powder |

The constituents of the basic material are heated to about 80° C. and mixed well. The pulverulent polymer PC is then incorporated into the basic mixture.

Formulation 16:

As Formulation 17, but instead of the pure powder PC, 0.5 g of Pigment Red 57:1 and 2.5 g of powder PC are incorporated.

Loose Powder Blusher (Formulations 17 to 19)

Formulation 17:

| | |
|---|---|
| 77.0 | talc |
| 10.0 | magnesium stearate |
| 2.0 | calcium carbonate |
| 0.5 | vaseline |
| 0.5 | paraffin oil |
| 10.0 | polymer PC as powder |

The dry powder constituents are homogeneously mixed and mixed with the molten and well-mixed fatty constituents.

Formulation 18:

As Formulation 17, although for a more intense red coloration, the pure pulverulent polymer PC can be replaced by a mixture of 1 to 2 g of red pigment, e.g. Pigment Red 172 Aluminium Lake and 8 to 9 g of pulverulent PC.

Formulation 19:

As Formulation 18, but using 9.5 g of pulverulent polymer PC and 0.5 g of iron oxide pigment.

W/O Type foundation

Formulation 20:

| | |
|---|---|
| 5.5 | hydrogenated castor oil, ethoxylated with 7 EO units |
| 7.0 | cetearyl octanoate |
| 4.5 | isopropyl myristate |
| 14.0 | paraffin oil |
| 0.3 | magnesium stearate |
| 0.3 | aluminum stearate |
| 2.0 | PEG-45/dodecyl glycol copolymer |
| 0.2 | propylparaben |
| 5.0 | propylene glycol |
| 0.6 | magnesium sulfate |
| 0.1 | paraben |
| 50.8 | water |
| 0.2 | perfume oil |
| 0.5 | vitamin E acetate |
| 9.0 | aqueous dispersion of PC (calculated as solid) |

The constituents of the fatty phase and of the water phase are heated separately to about 75° C., and the water phase is then slowly incorporated into the fatty phase with stirring. Following homogenization, the mixture is cooled to 40° C. with stirring, perfume oil and active ingredients are added, and the mixture is homogenized again. The aqueous dispersion of PC is then stirred in.

Formulation 21:

As Formulation 20, but using 8 g of polymer PC, 0.5 g of iron oxide pigment and 0.5 g of titanium dioxide pigment.

O/W Type Foundation

Formulation 22:

| | |
|---|---|
| 1.7 | glyceryl stearate |
| 1.7 | cetyl alcohol |
| 1.7 | ceteareth-6, stearyl alcohol |
| 1.7 | ceteareth-25 |
| 5.2 | caprylic/capric triglyceride |
| 0.2 | methyldibromoglutaronitrile (and/or) |
| | phenoxyethanol |
| 0.3 | imidazolidinylurea |
| 4.3 | propylene glycol |
| 69.0 | demineralized water |
| 0.2 | perfume oil |
| 14.0 | polymer PC as aqueous dispersion |

The constituents of the fatty phase and of the water phase are heated separately to about 75° C. The water phase, together with the aqueous dispersion of PC, is then slowly incorporated into the fatty phase with stirring. The mixture is homogenized and cooled with stirring to 40° C., perfume oil is added as desired, and the mixture is homogenized again.

Formulation 23:

As previous formulation, but using 12% of aqueous dispersion of PC, 1.5% of iron oxides and 0.5% of titanium dioxide.

Stage Make-Up

Formulation 24:

| | |
|---|---|
| 75.0 | petroleum distillate |
| 8.3 | quaternium-18 hectorite |
| 2.5 | propylene carbonate |
| 1.7 | polyvinylpyrrolidone/vinyl acetate copolymer |
| 12.5 | polymer PC as powder |

A gel is prepared from the constituents using strong shear forces. The copolymer and pulverulent polymer PC are incorporated. The mixture is then homogenized.

Formulation 25:

As previous formulation 24, but using 11 g of polymer PC and 1.5 g of conventional color pigment, e.g. Pigment Blue 15.

Formulation 26:

| | |
|---|---|
| 67.5 | mineral oil |
| 20.0 | beeswax |
| 10.0 | ceresin wax |
| 2.5 | polymer PC as powder |

Fatty components are melted and processed with pulverulent polymer PC to give a homogeneous paste.

Grease make-up for the stage in stick form

Formulation 27:

| | |
|---|---|
| 22.0 | ceresin wax |
| 18.0 | beeswax |
| 44.0 | mineral oil |
| 5.0 | turpentine |
| 1.0 | perfume oil |
| 8.0 | polymer PC as powder |
| 2.0 | iron hexacyanoferrate |

The fatty components are melted at 80° C., and the pulverulent polymer PC is mixed in. The composition is then perfumed and molded by casting or extrusion to give leads for cosmetic pencils.

Lipstick (Formulations 28 to 31)

Formulation 28:

| | |
|---|---|
| 3.0 | carnauba wax |
| 3.5 | candelilla wax |
| 2.0 | beeswax |
| 7.0 | microcrystalline wax |
| 1.5 | cetyl palmitate |
| 5.0 | vaseline |
| 3.5 | lanolin wax |
| 2.0 | lanolin |
| 9.0 | cetearyl octanoate |
| 0.2 | bisabolol |
| 0.5 | tocopherol |
| 2.0 | tocopheryl acetate |
| 3.5 | hydrogenated coconut fatty acid |
| | glyceride |
| 42.3 | castor oil |
| 15.0 | polymer PC as powder |

The constituents of the fatty composition are melted. The pulverulent polymer PC is then incorporated into the basic composition, and the homogeneous melt is poured into casting molds preheated to 60° C. The castings are removed from the molds while cold and, after warming to room temperature, are briefly flamed.

Formulation 29:

| | |
|---|---|
| 14.0 | oleyl alcohol |
| 10.0 | castor oil |
| 6.0 | diisopropyl adipate |
| 5.0 | stearamide MEA |
| 10.0 | polymer PC as powder |
| 1.0 | iron oxide pigment |
| 9.0 | stearyl heptanoate |
| 7.0 | isopropyl lanolate |
| 8.0 | carnauba wax |
| 10.0 | beeswax |
| 5.0 | cetyl alcohol |
| 5.0 | ozokerite |
| 3.0 | microcrystalline wax |
| 2.0 | polyethylene |
| 2.0 | petrolatum |
| 2.0 | mineral oil |
| 1.0 | perfume oil |

Formulation 30:

| | |
|---|---|
| 10.0 | hydroxyoctacosanyl hydroxystearate |
| 9.0 | candelilla wax |
| 25.0 | castor oil |
| 7.9 | isopropyl myristate |
| 5.0 | sorbitan trioleate |
| 3.0 | hydroxylated lanolin |
| 6.0 | butylene glycol |
| 0.1 | propylparaben |
| 1.0 | perfume oil |
| 3.0 | ultramarine |
| 30.0 | polymer PC as powder |

Formulation 31:

| | |
|---|---|
| 40.0 | castor oil |
| 10.0 | mineral oil |
| 9.0 | hydrogenated castor oil |
| 5.0 | cocoa butter |
| 10.0 | carnauba wax |
| 5.0 | stearyl heptanoate |
| 5.0 | beeswax |
| 10.0 | lanolin |
| 5.0 | polymer PC as powder |
| 1.0 | perfume oil |

Hair gel formulations (Formulations 32 to 34)
Formulation 32:

| | |
|---|---|
| 59.8 | water |
| 0.5 | polyacrylic acid (CTFA: Carbomer) |
| 1.2 | triethanolamine |
| 29.9 | glycerol |
| 2.0 | propylene glycol |
| 2.3 | dimethicone copolyol |
| 0.3 | imidazolidinylurea |
| 4.0 | polymer PC as aqueous dispersion |

Formulation 33:

| | |
|---|---|
| 0.7 | polyacrylic acid (CTFA: Carbomer) |
| 92.1 | water |
| 0.7 | hydrogenated castor oil, ethoxylated with 40 EO units |
| 0.2 | perfume oil |
| 0.3 | imidazolidinylurea |
| 1.0 | panthenol |
| 3.0 | polyvinylpyrrolidone |
| 1.0 | triethanolamine |
| 1.0 | polymer PC as aqueous dispersion |

Formulation 34 (styling gel):

| | |
|---|---|
| 0.5 | polyacrylic acid (CTFA: Carbomer) |
| 74.7 | water |
| 15.0 | ethanol |
| 0.2 | hydroxyethylcetyldimonium phosphate |
| 6.0 | polyvinylpyrrolidone |
| 0.3 | imidazolidinylurea |
| 0.8 | tetrahydroxypropylethylenediamine |
| 2.5 | polymer PC as aqueous dispersion |

Hair sprays (Formulations 35 to 37)
Formulation 35:

| | |
|---|---|
| 3.0 | polyvinylpyrrolidone |
| 4.0 | vinylpyrrolidone/vinyl acetate copolymer |
| 0.7 | rosin acrylate |
| 44.3 | ethanol |
| 3.0 | polymer PC as aqueous dispersion |
| 45.0 | propane/butane |

The components, with the exception of the aqueous dispersion of
the polymer PC, are dissolved. The aqueous dispersion of PC is
then stirred in. Prior to containerizing, add a few glass beads.

Formulation 36:

| | |
|---|---|
| 1.5 | acrylic acid/acrylamide copolymer |
| 0.11 | aminomethylpropanol |
| 0.02 | cyclomethicone |
| 6.0 | water |
| 3.0 | polymer PC as aqueous dispersion |
| 60.0 | dimethyl ether |
| 29.37 | ethanol |

Formulation 37:

The formulation corresponds to Formulation 36, but 2 g of the aqueous dispersion of PC and 1 g of Pigment Blue 15 are incorporated.

Hair Mascara (Formulations 38 to 40)

Formulation 38:

| | |
|---|---|
| 15.0 | mixture of beeswax, carnauba (Copernicia cerifera) wax, |
| | stearic acid, ceteareth-25, PEG-2 stearate SE, mineral oil, |
| | hydrogenated coconut oil and cetyl alcohol (Base RW 135, |
| | Wacker) |
| 1.5 | dimethicone |
| 0.5 | preservative |
| 42.1 | water |
| 0.45 | triethanolamine |
| 0.45 | xanthan, hectorite and cellulose gum |
| 30.0 | acrylic acid copolymer |
| 10.0 | polymer PC as aqueous dispersion |

Formulation 39:

As Formulation 38, but using 8 g of polymer PC and 2 g of Pigment
Blue 15.

Formulation 40:

| | |
|---|---|
| 14.0 | demin. water |
| 0.3 | imidazolidinylurea |
| 2.5 | Poloxamer 407 |
| 3.5 | polyvinylpyrrolidone |
| 11.0 | ethanol |
| 0.7 | triethanolamine |
| 0.52 | carbomer |
| 57.48 | demineralized water |
| 1.0 | iron oxide pigment |
| 9.0 | polymer PC as aqueous dispersion |

The components are formulated as gel, the color pigment and the
aqueous dispersion of PC being stirred in last.

Sunblock stick
Formulation 41:

| | |
|---|---|
| 4.0 | carnauba wax |
| 4.0 | candelilla wax |
| 4.0 | beeswax |
| 9.0 | microcrystalline wax |
| 1.0 | cetyl palmitate |
| 10.0 | lanolin wax |
| 5.0 | ethoxylated lanolin oil, 75 ethylene oxide units |
| 5.0 | cetearyl octanoate |
| 5.0 | octyl methoxycinnamate |
| 38.1 | caprylic/capric triglyceride |
| 0.2 | perfume oil |
| 2.0 | titanium dioxide |
| 0.5 | tocopherol |
| 2.0 | tocopheryl acetate |
| 0.2 | bisabolol |
| 5.0 | polymer PC with UV absorber as powder |

The constituents of the fatty composition are melted. Titanium dioxide is then stirred in. At 65° C., the active ingredients and the pulverulent polymer PC are incorporated into the basic composition. The homogeneous melt is poured into casting molds preheated to 60° C.

Colored Soap:

Formulation 42:

| | |
|---|---|
| 92.9 | soap flakes |
| 2.0 | polyquaternium-16 |
| 0.1 | bisabolol |
| 0.4 | tetrasodium EDTA |
| 2.0 | perfume oil |
| 1.0 | PEG-6 |
| 1.6 | water |

Incorporate 0.5 g of an aqueous dispersion of PC into 100 g of the basic soap composition comprising said constituents.

Formulation 43:

| | |
|---|---|
| 4.2 | sodium hydroxide |
| 5.6 | water |
| 22.6 | propylene glycol |
| 5.2 | cocoamide DEA |
| 10.4 | cocamine oxide |
| 4.2 | sodium lauryl sulfate |
| 7.3 | myristic acid |
| 16.6 | stearic acid |
| 5.2 | tocopheryl acetate |
| 18.7 | glycerol |

The ingredients are mixed and, at 85° C., are melted to give a clear melt. 100 parts of the basic soap composition are mixed with 3 parts of an aqueous dispersion of the polymer PC, and the resulting composition is poured into molds while still hot.

Color-Imparting Hair Foam (Formulations 44 and 45)

Formulation 44:

| | |
|---|---|
| 2.0 | cocotrimonium methosulfate |
| 0.2 | perfume oil |
| 7.0 | polyquaternium-64 |
| 2.0 | polyquaternium-11 |
| 0.2 | ceteareth 25 |
| 0.5 | panthenol |
| 0.05 | benzophenone-4 |
| 0.2 | mixture of amodimethicone, tallow trimonium |
| | chloride and nonoxynol 10 |
| 0.2 | hydroxyethylcellulose |
| 15.0 | ethanol |
| 1.5 | polymer PC as aqueous dispersion |
| 10.0 | propane/butane |
| | water ad 100 g |

The components are mixed and containerized together with the propellant.
Formulation 45:

| | |
|---|---|
| 2.0 | cocotrimonium methosulfate |
| 0.2 | perfume oil |
| 6.7 | acrylic acid copolymer |
| 0.6 | aminomethylpropanol |
| 2.5 | polyvinylcaprolactam |
| 0.2 | ceteareth 25 |
| 0.2 | panthenol |
| 0.1 | PEG-25 PABA |
| 0.2 | hydroxyethylcellulose |
| 15.0 | ethanol |
| 1.0 | polymer PC as aqueous dispersion |
| 10.0 | propane/butane |
| | water ad 100 g |

Colored hair shampoo
Formulation 46:

| | |
|---|---|
| 40.0 | sodium lauryl sulfate |
| 10.0 | cocoamidopropylbetaine |
| q.s. | perfume oil |
| 3.0 | polyquaternium-44 |
| q.s. | preservative |
| 0.5 | sodium chloride |
| 1.5 | polymer PC as aqueous dispersion |
| | water ad 100 g |

Sunscreen cream
Formulation 47:

| | |
|---|---|
| 1.5 | ceteareth 6 |
| 1.0 | cetanol |
| 3.0 | cetearyl octanoate |
| 5.0 | polymer powder from example 1 |
| 2.0 | butylmethoxydibenzoylmethane |
| 6.0 | isopropyl stearate |
| 1.0 | glyceryl stearate |
| 2.0 | stearic acid |
| 3.0 | polyethylene glycol 300 |
| 0.3 | carbomer |
| 0.6 | tetrahydroxypropylethylenediamine |
| 0.1 | disodium EDTA |
| 0.1 | butylparaben |
| 0.2 | methylparaben |
| 74.2 | water |

We claim:

1. A colorant-containing aqueous polymer dispersion comprising:
a. colorant-containing polymer particles PC having an average particle diameter d₅₀ below 1000 nm, comprising:
i. a polymer matrix constructed from ethylenically unsaturated monomers M and
ii. at least one organic colorant C homogeneously distributed within the polymer matrix and chosen from dyes, UV absorbers and optical brighteners, in an amount of from 0.5 to 50% by weight, based on the polymer matrix;
b. at least one nonionic surface-active compound NS in an amount of from 0.1 to 20% by weight, based on the polymer matrix; and
c. at least one amphiphilic polymer PA which has 0.5 to 10 mol/kg of anionic functional groups, in an amount of from 1 to 50% by weight, based on the polymer matrix.

2. A polymer dispersion as claimed in claim 1, in which the nonionic surface-active compound has at least one alkyl group having 6 to 32 carbon atoms and at least one oligoether group of the formula —[CH₂CH₂O]ₙ—H, in which n is at least 2.

3. A polymer dispersion as claimed in claim 1, in which the amphiphilic polymer PA has carboxylate groups as anionic functional groups.

4. A polymer dispersion as claimed in claim 3, in which the polymer PA comprises, in copolymerized form:
5 to 60 mol % of at least one monomer A, chosen from monoethylenically unsaturated mono- and dicarboxylic acids having 3 to 8 carbon atoms;
40 to 95 mol % of at least one monomer B which is insoluble or has limited solubility in water, and optionally
up to 30 mol % of a monomer C which is different from monomers A and B, in each case based on the sum of the monomers A, B and C,
and in which up to 50 mol % of the carboxyl groups can be esterified by a surface-active compound with oligoether group of the formula —[CH₂CH₂O]ₙ—H, in which n is at least 2.

5. A polymer dispersion as claimed in claim 4, in which the polymer PA is chosen from the salts of:
copolymers of maleic acid as monomer A and C₃-C₂₀-olefins as monomer B, in which 1 to 50 mol % of the carboxyl groups can be esterified by a surface-active compound containing oligoether groups of the formula —[CH₂CH₂O₃]ₙ—H, in which n is at least 2;
copolymers of monoethylenically unsaturated C₃-C₈-monocarboxylic acids as monomer A with vinyl esters of aliphatic C₂-C₂₀-carboxylic acids as monomer B;
copolymers of monomers A, chosen from the C₁-C₂₀-alkyl esters of monoethylenically unsaturated monocarboxylic acids and the N-C₁-C₂₀-alkyl- and N,N-(di-C₁-C₂₀-alkyl)amides of monoethylenically unsaturated C₃-C₈-monocarboxylic acids.

6. A polymer dispersion as claimed in claim 1, in which the polymer matrix comprises, as monomers M:
70 to 99.9% by weight of at least one monomer M1 having a solubility in water in the range from 0.01 g/l to 80 g/l (at 25° C. and 1 bar),
0.1 to 30% by weight of at least one crosslinking monomer M2 which has at least two nonconjugated ethylenically unsaturated double bonds, and
0 to 20% by weight of one or more monomers M2 having a solubility in water of <0.01 g/l (at 25° C. and 1 bar),
0 to 30% by weight of monomers M4 different therefrom, where the weight proportions of the monomers M1 to M4 are in each case based on 100% by weight of monomers M.

7. A polymer dispersion as claimed in claim 1, in which the colorant C is a UV absorber.

8. A colorant-containing polymer powder obtainable by evaporating the volatile constituents of an aqueous colorant-containing polymer dispersion as defined in claim 1.

9. A process for the preparation of a colorant-containing polymer dispersion as defined in claim 1, comprising the successive steps:
i. dissolution of the colorant C in the monomers M,
ii. production of a conventional, colorant-containing oil-in-water emulsion by emulsifying the monomer/colorant solution in water and p1 iii. homogenization of the conventional emulsion to give a colorant-containing miniemulsion in which the monomer droplets have an average droplet diameter below 1000 nm,
iv. polymerization of the miniemulsion in the presence of a polymerization initiator which triggers the free-radical polymerization of the monomers M,
which comprises carrying out the polymerization in the presence of from 0.1 to 20% by weight of at least one nonionic surface-active compound NS and 1 to 50% by weight, in each case based on the monomers M, of at least one amphiphilic polymer PA.

10. A cosmetic composition comprising at least one colorant-containing polymer PC in the form of an aqueous polymer dispersion as defined in claim 1 and/or a polymer powder as defined in claim 8 and adjuvants customary for cosmetic compositions.

11. A cosmetic composition in the form of a sunscreen formulation, comprising at least one UV-absorber-containing polymer PC in the form of an aqueous polymer dispersion as defined in claim 7 and/or a polymer powder prepared therefrom.
